(19) United States
(12) Patent Application Publication
(10) Pub. No.: US 2021/0179963 A1
(43) Pub. Date: Jun. 17, 2021

(54) LUBRICANT FOR MEDICAL DEVICE AND MEDICAL DEVICE

(71) Applicant: OLYMPUS CORPORATION, Tokyo (JP)

(72) Inventor: Minoru HARA, Yokohama-shi (JP)

(73) Assignee: OLYMPUS CORPORATION, Tokyo (JP)

(21) Appl. No.: 17/190,067

(22) Filed: Mar. 2, 2021

Related U.S. Application Data
(63) Continuation of application No. PCT/JP2019/031797, filed on Aug. 13, 2019.

(30) Foreign Application Priority Data
Oct. 9, 2018 (JP) 2018-191189

(51) Int. Cl. C10M 169/04; A61B 1/005; C10M 129/14; C10M 129/24; C10M 103/06

(52) U.S. Cl. CPC C10M 169/04; A61B 1/005; C10N 2040/50; C10M 129/24; C10M 103/06; C10M 129/14

(57) ABSTRACT

A lubricant for a medical device includes an anti-friction material and a radical scavenger.

The application is a continuation application based on a PCT Patent Application No. PCT/JP2019/031797, filed Aug. 13, 2019, whose priority is claimed on Japanese Patent Application No. 2018-191189, filed Oct. 9, 2018. The contents of both the PCT Application and the Japanese Application are incorporated herein by reference.

### BACKGROUND OF THE INVENTION

### Field of the Invention

The present invention relates to a lubricant for a medical device and the medical device.

### Description of Related Art

In recent years, gas low-temperature sterilization has been widely used as sterilization treatment for a medical device. For example, hydrogen peroxide gas is often used as sterilization gas in the gas low-temperature sterilization.

Examples of a medical device to be subjected to sterilization treatment include devices, such as an endoscope that is used while being inserted into the body, and endoscopic devices that are used together with an endoscope. In such a medical device, tubular members or shaft-like members are movably inserted into a flexible tube. A lubricant is used in order to facilitate the movement of the tubular members or the shaft-like members in the flexible tube. The lubricant reduces friction between the inner circumference surface of the flexible tube and the tubular members or the shaft-like members.

However, there is a possibility that sterilization gas may chemically react with not only bacteria adhering to the medical device but also a lubricant and each member of the medical device.

Particularly, lubricants for a medical device often include molybdenum disulfide. Molybdenum disulfide is a solid anti-friction material. Sulfur components contained in molybdenum disulfide are likely to chemically react with sterilization gas components in a gas low-temperature sterilization process. For example, sulfurous acid, sulfuric acid, and the like are generated in a case where molybdenum disulfide chemically reacts with hydrogen peroxide. As a result, the resin, metal, and the like of the respective members of a medical device deteriorate or corrode.

For example, Japanese Unexamined Patent Application, First Publication No. H11-318814 discloses a technique in which a material having a catalytic action on hydrogen peroxide or the low-temperature plasma of hydrogen peroxide is used for a structural member of an insertion portion of an endoscope in order to improve the resistance of the insertion portion of the endoscope to hydrogen peroxide.

In the Japanese Unexamined Patent Application, examples of a material having a catalytic action on the low-temperature plasma of hydrogen peroxide include silver, copper, nickel, palladium, and platinum.

### SUMMARY OF THE INVENTION

A lubricant for a medical device of a first aspect of the invention includes an anti-friction material and a radical scavenger.

A medical device of a second aspect of the invention includes the lubricant for a medical device.

### BRIEF DESCRIPTION OF THE DRAWINGS

FIG. 1 is a schematic perspective view showing the schematic configuration of an endoscope that is an example of a medical device according to an embodiment of the present invention.

FIG. 2 is a schematic cross-sectional view of an insertion portion of the endoscope that is an example of the medical device according to the embodiment of the present invention.

FIG. 3 is a schematic enlarged view of a portion A of FIG. 2.

### DETAILED DESCRIPTION OF THE INVENTION

A lubricant for a medical device and the medical device of embodiments of the present invention will be described below with reference to the accompanying drawings.

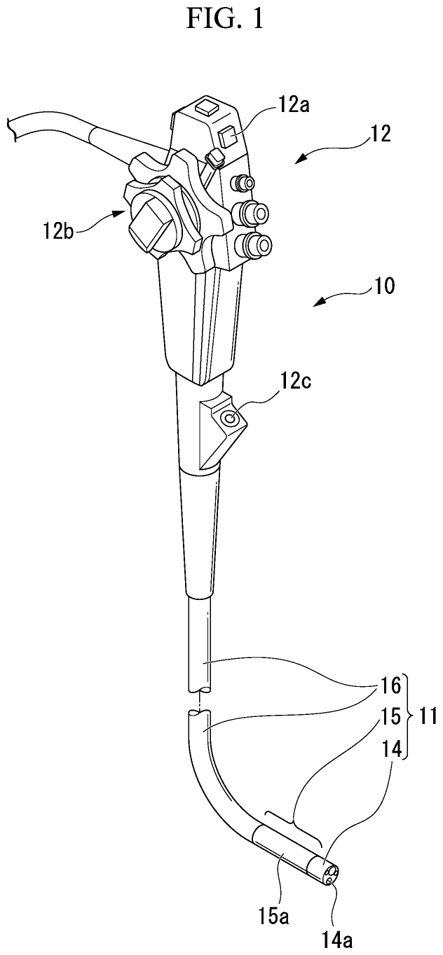

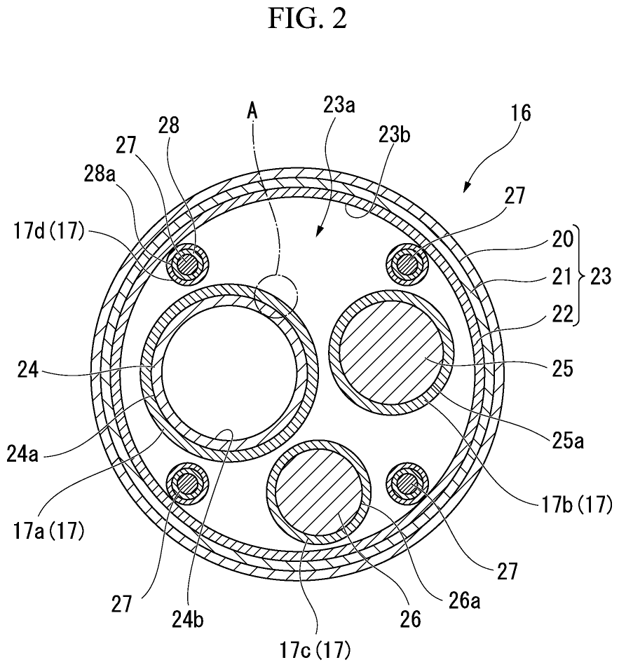

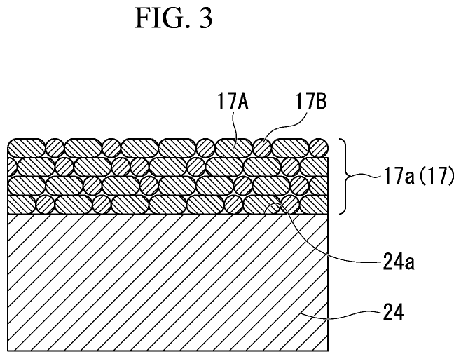

FIG. 1 is a schematic perspective view showing the schematic configuration of an endoscope that is an example of a medical device according to an embodiment of the present invention. FIG. 2 is a schematic cross-sectional view of an insertion portion of the endoscope that is an example of the medical device according to the embodiment of the present invention. FIG. 3 is a schematic enlarged view of a portion A of FIG. 2.

An endoscope 10 (medical device) of the present embodiment shown in FIG. 1 is a medical endoscope that is used while being inserted into the body of a patient. Sterilization treatment to be applied to the endoscope 10 is gas low-temperature sterilization. The type of gas low-temperature sterilization treatment is not particularly limited. Examples of gas low-temperature sterilization treatment suitable for the endoscope 10 include hydrogen peroxide low-temperature plasma sterilization, hydrogen peroxide gas low-temperature sterilization, ethylene oxide gas sterilization, and the like.

The endoscope 10 includes an insertion portion 11 and an operating portion 12.

The insertion portion 11 is formed in the form of a flexible tube in order to be inserted into the body of a patient. The insertion portion 11 includes a distal end portion 14, a bending portion 15, and a flexible tube portion 16 that are arranged in this order from the distal end in an insertion direction. Although not shown in FIG. 1, an endoscopic channel (to be described later) extending in the longitudinal direction of the insertion portion 11 is disposed in the insertion portion 11 so that an endoscopic device is inserted into the endoscopic channel.

The distal end portion 14 is disposed at a portion that includes the most distal end of the endoscope 10. The distal end portion 14 includes an end effector of the endoscope 10 that functions as a manipulator. For example, in the present embodiment, an imaging device, such as a CCD, and an imaging optical system including an appropriate lens are provided in the distal end portion 14 in order to acquire the image of an object to be investigated. In the present embodiment, the distal end portion 14 has a columnar appearance.

The imaging device is disposed on the image surface of the imaging optical system. The imaging device photoelectrically converts received light to generate image signals.

The image signals generated by the imaging device are transmitted to the operating portion 12 to be described later through metal wires. The image signals may be subjected to signal processing as necessary before being transmitted to the operating portion 12.

The metal wires include a signal line and a power line. The signal line supplies a control signal to the imaging device. The power line supplies a drive voltage to the imaging device. The metal wires are put together in a cable.

However, the imaging device may be disposed in the operating portion 12 to be described later. In this case, the distal end of an image guide, which transmits a light image to the imaging device, is disposed on the image surface of the imaging optical system. The image guide extends up to the operating portion 12, in which the imaging device is disposed, via the inside of the bending portion 15 and the flexible tube portion 16 to be described later. A bundle of optical fibers may be used as the image guide.

An image acquired by the distal end portion 14 is transmitted as image signals or image light through the metal wires or the bundle of optical fibers in the bending portion 15 and the flexible tube portion 16 to be described later. The metal wires or the bundle of optical fibers form a linear image transmission cable.

The distal end of the distal end portion 14 is provided with an imaging window, an illumination window, and an opening 14a. The opening 14a communicates with an endoscopic channel to be described later.

The bending portion 15 is connected to the proximal end of the distal end portion 14. The bending portion 15 is a tubular portion that is adapted to be bendable in order to change the direction of the distal end portion 14.

The bending portion 15 includes, for example, a plurality of annular nodal rings. The plurality of nodal rings are rotatably connected to each other. Operating wires to be described later are inserted into the plurality of nodal rings.

For example, linear members, such as electrical wires connected to the imaging device of the distal end portion 14 and a fiber light guide extending up to the illumination window, are accommodated in the bending portion 15.

The linear members, such as the operating wires, the image transmission cable, and the fiber light guide having been described above, are inserted into the flexible tube portion 16 to be described later and extend up to the operating portion 12 to be described later.

The bending portion 15 is covered with an outer tube 15a.

The flexible tube portion 16 is a tubular portion that connects the bending portion 15 to the operating portion 12 to be described later.

As shown in a cross-section in FIG. 2, the flexible tube portion 16 includes a flexible tube 23. A lumen 23a penetrates the flexible tube 23 in the longitudinal direction of the flexible tube 23. Long built-in elements, such as an endoscopic channel 24 (insertion member), an image transmission cable 25 (insertion member), a fiber light guide 26 (insertion member), and operating wires 27 are inserted into the lumen 23a of the flexible tube 23.

The flexible tube 23 includes a flex 22, a stainless steel blade 21, and an outer tube 20. The flex 22, the stainless steel blade 21, and the outer tube 20 are arranged in this order from the inner circumference portion of the flexible tube 23 toward the outer circumference portion thereof.

The flex 22 is formed of a belt-like member that is made of, for example, metal or a resin and is helically wound. The inner circumference surface of the flex 22 forms an inner circumference surface 23b of the flexible tube 23. The lumen 23a is a space inside the inner circumference surface 23b.

The stainless steel blade 21 is formed in the form of a net-like tube that is woven with a stainless steel wire. The stainless steel blade 21 covers the flex 22 from the outer circumference side. The stainless steel blade 21 overlaps the flex 22.

The outer tube 20 is a tubular member made of a soft resin. The outer tube 20 covers the stainless steel blade 21 from the outer circumference side. The outer tube 20 overlaps the stainless steel blade 21.

According to this structure, the flexible tube 23 can be bent in an appropriate direction in a state where the flexible tube 23 maintains a substantially circular cross-section.

The endoscopic channel 24 is a tubular member that forms an insertion passage into which an appropriate medical device, for example, an endoscopic device, a catheter, and the like, can be inserted. The distal end of the endoscopic channel 24 penetrates the distal end surface of the distal end portion 14 (see FIG. 1). The distal end of the endoscopic channel 24 forms an opening through which an endoscopic device, a catheter, and the like come in and out.

The distal end of the endoscopic channel 24 communicates with the opening 14a (see FIG. 1).

The proximal end of the endoscopic channel 24 is connected to a forceps valve 12c (see FIG. 1) provided on the operating portion 12 to be described later.

The endoscopic channel 24 is formed of, for example, a flexible resin tube. The endoscopic channel 24 can be bent together with the flexible tube portion 16. It is more preferable that a material allowing an endoscopic device, a catheter, and the like being in contact with an inner circumference surface 24b of the endoscopic channel 24 to easily slide is selected as the resin material of the endoscopic channel 24.

For example, a polyethylene resin, a fluorine-based resin, a urethane-based resin, and the like may be used as the material of the endoscopic channel 24.

The image transmission cable 25 transmits an image, which is acquired by the imaging optical system of the distal end portion 14, to the operating portion 12 as image signals or image light. For example, in a case where the image transmission cable 25 transmits image signals, a linear body including a metal wire covered with a flexible resin tube is used as the image transmission cable 25. For example, in a case where the image transmission cable 25 transmits image light, a linear body including an optical fiber covered with a flexible resin tube is used as the image transmission cable 25.

The fiber light guide 26 supplies illumination light. The illumination light is supplied from the illumination window of the distal end portion 14 in order to illuminate the outside. A structure where an optical fiber transmitting illumination light is covered with a flexible resin tube is used as the fiber light guide 26.

The distal end of the fiber light guide 26 is disposed so as to face the illumination window of the distal end portion 14. The fiber light guide 26 extends into the flexible tube 23 via the distal end portion 14 and the bending portion 15. The proximal end of the fiber light guide 26 is optically coupled to a light source disposed in the operating portion 12 to be described later.

The operating wires 27 transmit a driving force for bending the bending portion 15. For example, in a case where the bending portion 15 is adapted to be bendable in two axis directions, four operating wires 27 are provided as shown in FIG. 2. Each of the distal ends of the operating wires 27 is connected to a connection member (not shown) provided at the distal end of the bending portion 15. The operating wires 27 are divided into positions that face each other with a central axis of the bending portion 15 interposed therebetween in two directions orthogonal to the central axis in the bending portion 15 and are inserted into the nodal rings.

Each operating wire 27 is inserted into a coil sheath 28 (insertion member) for the purpose of maintaining a constant path length in the flexible tube 23 even though the flexible tube 23 is bent.

Each coil sheath 28 has a structure where a metal wire is closely wound. Each coil sheath 28 has an inner diameter substantially equal to the outer diameter of the operating wire 27.

The coil sheaths 28 are inserted into the flexible tube portion 16. The coil sheaths 28 cover the operating wires 27 from the outer circumference side.

The distal ends of the respective coil sheaths 28 are fixed to a cap (not shown) provided at the proximal end of the bending portion 15. The proximal ends of the respective coil sheaths 28 are fixed to a fixing plate (not shown) provided in the operating portion 12.

Each coil sheath 28 is not particularly fixed in the flexible tube 23. As a result, each coil sheath 28 can be moved in a gap formed in the flexible tube 23. However, the entire length of each coil sheath 28 is not changed even though each coil sheath 28 is moved or bent in the flexible tube 23.

The endoscopic channel 24, the image transmission cable 25, the fiber light guide 26, and the coil sheaths 28 are accommodated in the flexible tube 23. The endoscopic channel 24, the image transmission cable 25, the fiber light guide 26, and the coil sheaths 28 are almost parallel to each other in the flexible tube 23. Each of the endoscopic channel 24, the image transmission cable 25, the fiber light guide 26, and the coil sheaths 28 is a flexible linear insertion member.

In a case where the flexible tube 23 is bent, each insertion member is also deformed depending on the deformation of the flexible tube 23. The respective insertion members slide on each other while being in contact with each other, or slide on the inner circumference surface 23b of the flexible tube 23 while being in contact with the inner circumference surface 23b. In this case, a friction force acts between each insertion member and the flexible tube 23. As a result, a deformation load corresponding to the magnitude of a friction force is generated in a case where the flexible tube 23 is deformed. Since the flexible tube portion 16 cannot be smoothly inserted into the body of a patient in a case where the deformation load is increased, a burden on not only an operator but also a patient is increased.

A lubricant layer 17 (a lubricant) is formed on the surface of each insertion member in the present embodiment. In a case where the lubricant layers 17 of the respective insertion members are to be distinguished from each other in the following description, lowercase alphabet letters a, b, c, and d are added for distinguishment. A lubricant layer 17a is a lubricant layer 17 that is formed on an outer circumference surface 24a of the endoscopic channel 24. A lubricant layer 17b is a lubricant layer 17 that is formed on an outer circumference surface 25a of the image transmission cable 25. A lubricant layer 17c is a lubricant layer 17 that is formed on an outer circumference surface 26a of the fiber light guide 26. A lubricant layer 17d is a lubricant layer 17 that is formed on an outer circumference surface 28a of each coil sheath 28.

However, an adherend on which the lubricant layer 17 is to be formed is not limited to the respective insertion members having been described above.

For example, the lubricant layer 17 may be formed on the surfaces of other insertion members (not shown) in the lumen 23a of the flexible tube 23. For example, the lubricant layer 17 may be disposed on the inner circumference surface 23b. For example, part of each lubricant layer 17 may adhere to the other members provided in the lumen 23a so as to be disposed in the lumen 23a.

In addition, the lubricant layer 17 may be disposed on any member as long as the member is apart of the endoscope 10. For example, the lubricant layer 17 may be disposed on the surfaces of appropriate device bodies that slide on each other in the endoscope 10.

The specific structure of the lubricant layer 17 will be described after the description of the operating portion 12.

As shown in FIG. 1, the operating portion 12 is part of the device that is used by an operator in order to operate the endoscope 10. Examples of an operation using the operating portion 12 can include an operation for pulling the operating wires 27 in order to change the amount of bending of the bending portion 15. The operating portion 12 includes, for example, an operation switch 12a, operation knobs 12b, and the like.

The forceps valve 12c is provided on the distal end side of the operating portion 12 in order to allow an endoscopic device, a catheter, and the like to be inserted into the endoscopic channel 24. The forceps valve 12c includes a valve body that prevents the back flow of fluid in the endoscopic channel 24. As a result, the back flow of fluid in the endoscopic channel 24 is prevented in a case where an endoscopic device, a catheter, and the like are inserted and removed through the forceps valve 12c.

As schematically shown in FIG. 2, the respective lubricant layers 17 are provided in the form of a layer on the outer circumference surface 24a of the endoscopic channel 24, the outer circumference surface 25a of the image transmission cable 25, the outer circumference surface 26a of the fiber light guide 26, and the outer circumference surfaces 28a of the coil sheaths 28, respectively. The endoscopic channel 24, the image transmission cable 25, the fiber light guide 26, and the coil sheaths 28 form part of the device body of the endoscope 10. The endoscopic channel 24, the image transmission cable 25, the fiber light guide 26, and the coil sheaths 28 are the adherends for the lubricant layers 17.

In the present embodiment, the adherends for the lubricant layers 17a, 17b, 17c, and 17d are different from each other but the lubricant layers 17a, 17b, 17c, and 17d have the same structure. The structure of the lubricant layer 17 will be described below using the lubricant layer 17a as an example. The following description of the lubricant layer 17a is also applied to the lubricant layers 17b, 17c, and 17d likewise except for a difference in adherend.

The lubricant layer 17a provided on the outer circumference surface 24a of the endoscopic channel 24 is schematically shown in FIG. 3.

As schematically shown in FIG. 3, the lubricant layer 17a has an anti-friction material 17A and a radical scavenger 17B and is provided in the form of a layer on the outer circumference surface 24a. In the present embodiment, the anti-friction material 17A and the radical scavenger 17B are substantially uniformly mixed in the lubricant layer 17a.

Appropriate additives, for example, inorganic fillers, organic fillers, and the like may be contained in the lubricant layer 17a in addition to the anti-friction material 17A and the radical scavenger 17B. Moreover, an ion exchanger may be included in the lubricant layer 17a in addition to the anti-friction material 17A and the radical scavenger 17B.

The thickness of the lubricant layer 17a is not particularly limited as long as a friction reduction effect required for the endoscopic channel 24 is obtained. For example, an appropriate thickness may be determined as the thickness of the lubricant layer 17a in consideration of the stability of adhesion of the anti-friction material 17A and the radical scavenger 17B to the outer circumference surface 24a.

In addition, the thickness of the lubricant layer 17a does not need to be constant. As long as a required friction reduction effect can be obtained, the lubricant layer 17a may not cover part of the outer circumference surface 24a.

The layered structure schematically shown in FIG. 3 is exemplary. The layered structure of the lubricant layer 17a is not limited to the layered structure shown in FIG. 3.

For example, the lubricant layer 17a shown in FIG. 3 is drawn so that the anti-friction material 17A and the radical scavenger 17B are multiply stacked in a thickness direction. However, the thicknesses of the anti-friction material 17A and the radical scavenger 17B do not need to be equal to each other as shown in FIG. 3. The lubricant layer 17a may be a layered body of a mixture of the anti-friction material 17A and the radical scavenger 17B.

The anti-friction material 17A and the radical scavenger 17B are disposed on the outer circumference surface 24a in a state where the anti-friction material 17A and the radical scavenger 17B are exposed to the lumen 23a. It is more preferable that the anti-friction material 17A and the radical scavenger 17B are closely adjacent to each other. However, the anti-friction material 17A and the radical scavenger 17B may be away from each other. The anti-friction material 17A and the radical scavenger 17B may be distributed in the shape of an island larger than the particle sizes thereof and may be adjacent to each other or away from each other with the shape of an island as a unit.

Even though the anti-friction material 17A and the radical scavenger 17B are multiply stacked in the thickness direction as shown in FIG. 3, the anti-friction material 17A and the radical scavenger 17B are mixed with each other and dispersed according to the percentage contents thereof as seen in the thickness direction. As a result, both the anti-friction material 17A and the radical scavenger 17B are exposed to the surface of the lubricant layer 17a.

An appropriate solid lubricant not affecting the durability of an adherend, such as the endoscopic channel 24, is used as the material of the anti-friction material 17A. Examples of the solid lubricant suitable for the anti-friction material 17A include molybdenum disulfide (MoS₂), graphite, fluororesin particles, graphite fluoride, boron nitride, and the like. Examples of the fluororesin particles include polytetrafluoroethylene (PTFE), PFA (a copolymer of tetrafluoroethylene (C₂F₄) and perfluoroalkoxyethylene), and the like.

The anti-friction material 17A may be formed of one type of solid lubricant, and may be formed of a mixture of a plurality of types of solid lubricants.

The radical scavenger 17B is a material that deactivates a radical. A radical is also referred to as a free radical. The radical scavenger 17B is used in order to improve the sterilization resistance of an adherend for the anti-friction material 17A or the lubricant layer 17.

The inventor has investigated in earnest for the further improvement of the sterilization resistance of the anti-friction material 17A and an adherend in gas low-temperature sterilization treatment using sterilization gas. The inventor has newly found that the sterilization resistance of the anti-friction material 17A and an adherend can be significantly improved in a case where the lubricant layer 17 is formed through a combination of the anti-friction material 17A and a radical scavenger. As a result, the inventor has reached the present invention.

The mechanism of the action of the sterilization gas in the gas low-temperature sterilization is complex. Accordingly, it is not considered that only the presence of a radical in the sterilization gas contributes to a chemical reaction related to sterilization in the gas low-temperature sterilization. However, according to the inventor's investigation, in a case where a radical scavenger is contained in the lubricant layer 17, better sterilization resistance is obtained as compared to metal particles that are said to have a catalytic action on the sterilization gas.

The type of the radical scavenger 17B is not particularly limited as long as the radical scavenger is a material that can deactivate a radical generated in gas low-temperature sterilization. Examples of a radical generated in the case of hydrogen peroxide gas sterilization include an oxygen radical, a hydroxyl radical, and the like.

Examples of a compound suitable for the radical scavenger 17B include one or more compounds selected from a group consisting of hydroquinone, benzoquinone, 4-tert-butylpyrocatechol, tert-butylhydroquinone, 2-tert-butyl-4,6-dimethylphenol, butylhydroxytoluene, 2,6-di-tert-butylphenol, and hydroquinone monomethyl ether. Hydroquinone is also called 1,4-benzenediol, p-benzenediol, or the like.

The isomers of benzoquinone include 1,4-benzoquinone (p-benzoquinone) and 1,2-benzoquinone (o-benzoquinone). At least one of 1,4-benzoquinone and 1,2-benzoquinone can be used as the benzoquinone in the radical scavenger 17B.

Particularly, it is more preferable that at least one of hydroquinone and benzoquinone is contained in the radical scavenger 17B.

The content of the anti-friction material 17A in the lubricant layer 17a(17) may be determined depending on friction reduction effect required for the insertion member. Hereinafter, the content of the anti-friction material 17A and the content of the radical scavenger 17B represent the contents thereof in the lubricant layer 17a(17) as long as being not particularly specified.

For example, it is more preferable that a coefficient of dynamic friction between each insertion member and the flexible tube 23 is 0.500 or less in the case of the endoscope 10. For example, it is still more preferable that a coefficient of dynamic friction between each insertion member and the flexible tube 23 is 0.470 or less in the case of the endoscope 10.

The content of the radical scavenger 17B may be determined depending on durability of the lubricant layer 17a(17) regarding to the friction reduction effect that is required for the insertion member. For example, as durability required for the endoscope 10, it is more preferable that a coefficient of dynamic friction is 0.500 or less even after gas low-temperature sterilization is performed 200 or more times.

Specifically, the content of the radical scavenger 17B may be 0.1 mass % or more and 70 mass % or less. In this case, the content of the anti-friction material 17A may exceed 30 mass % and may be less than 99.9 mass %.

It is more preferable that the content of the radical scavenger 17B is 10 mass % or more and 70 mass % or less. In this case, it is more preferable that the content of the anti-friction material 17A exceeds 30 mass % and is less than 90 mass %.

There is a concern that it may be difficult for a chemical reaction between the sterilization gas and the anti-friction material 17A to be suppressed in a case where the content of the radical scavenger 17B is less than 0.1 mass %.

There is a concern that the friction reduction effect of the lubricant layer 17a may be reduced due to a relative reduction in the content of the anti-friction material 17A in a case where the content of the radical scavenger 17B exceeds 70 mass %.

For example, in a case where the radical scavenger 17B is made of benzoquinone, the content of benzoquinone may be 0.1 mass % or more and 70 mass % or less.

For example, in a case where the radical scavenger 17B is made of hydroquinone, the content of hydroquinone may be 10 mass % or more and 70 mass % or less.

The above-mentioned lubricant layer 17 can be manufactured, for example, as follows.

First, a material to be applied is prepared in order to form the lubricant layer 17. At least the anti-friction material 17A and the radical scavenger 17B are mixed with each other to manufacture the material to be applied. The above-mentioned additives and the like may be contained in the material to be applied in addition to the anti-friction material 17A and the radical scavenger 17B.

Then, the material to be applied is applied to the surface of an adherend. A dry application method or a wet application method is used as a method of applying the material to be applied.

Examples of the dry application method include spray application, rubbing application, and the like. In the case of the rubbing application, for example, a material to be applied may be rubbed on the surface of an adherend while a pressing force is applied to the material to be applied by, for example, an application jig, a hand, or the like. In the case of the rubbing application, for example, a material to be applied adhering to the surface of an adherend may be swept along the surface of the adherend by an application jig, a hand, or the like.

As the wet application method, dispersed liquid to be applied in which a material to be applied is dispersed in a solution to be applied may be formed and may be then applied to an adherend by, for example, spraying, dipping, or the like. After that, for example, the solution to be applied is evaporated by the heating of the adherend or the like, so that the lubricant layer 17 is formed on the surface of the adherend.

In this way, the lubricant layers 17a, 17b, 17c, and 17d are formed on the surfaces of the insertion members formed of the endoscopic channel 24, the image transmission cable 25, the fiber light guide 26, and the coil sheaths 28, respectively.

Each insertion member on which the lubricant layer 17 is formed is inserted into the flexible tube 23 as shown in FIG. 2. The insertion members are fixed to fixing counterpart members at fixing positions, respectively. The operating wires 27 are inserted into the coil sheaths 28, respectively.

The endoscope 10 is manufactured as described above.

Next, the action of the lubricant layer 17 will be mainly described with regard to the action of the endoscope 10.

The endoscope 10 is a medical device that is used after being subjected to gas low-temperature sterilization. The endoscope 10 is repeatedly subjected togas low-temperature sterilization.

In the gas low-temperature sterilization, microorganisms to be subjected to sterilization chemically react with reactive components caused by the sterilization gas and are thus destroyed. However, the reactive components caused by the sterilization gas also chemically attack the structural members of the endoscope 10. As a result, there is a concern that the reactive components caused by the sterilization gas may cause the structural members to deteriorate.

Examples of the reactive components caused by the sterilization gas include ions that are ionized by the sterilization gas, radicals that are generated due to the sterilization gas, highly reactive intermediates that are generated in a sterilization process, and the like.

According to the lubricant layer 17, since the anti-friction material 17A and the radical scavenger 17B are mixed with each other, the deterioration of the anti-friction material 17A in a sterilization process is significantly suppressed.

The mechanism of a reaction in a sterilization process is complex. Accordingly, the specific action of the radical scavenger 17B is not specified with regard to an action for suppressing the deterioration of the anti-friction material 17A. However, it is considered that at least radicals likely to react with a compound forming the anti-friction material 17A are deactivated by the radical scavenger 17B positioned near the anti-friction material 17A due to the action of the radical scavenger 17B.

For example, in a case where molybdenum disulfide is contained in the anti-friction material 17A and hydrogen peroxide is used as the sterilization gas, the hydrogen peroxide is chemically combined with sulfur components of the molybdenum disulfide and sulfurous acid and sulfuric acid are generated. In a case where part of the molybdenum disulfide is consumed by a reaction, the lubricity of the anti-friction material 17A is reduced due to the destruction of molecular structure having lubricity. In addition, reaction products, such as sulfurous acid and sulfuric acid, cause the structural members of the endoscope 10 to corrode.

The radical scavenger 17B can suppress the chemical reaction of molybdenum disulfide that is caused by radicals generated during the gas low-temperature sterilization. As a result, the radical scavenger 17B can prevent a reduction in the lubricity of molybdenum disulfide and the deterioration of the structural members of the endoscope 10 caused by reaction products.

Even in a case where molybdenum disulfide is not contained in the anti-friction material 17A, the chemical structure of the anti-friction material 17A is damaged due to a reaction between the anti-friction material 17A and radicals. As a result, it is considered that the friction reduction action of the anti-friction material 17A deteriorates. In this case, even though reaction products generated due to a reaction between the anti-friction material 17A and radicals do not cause the structural members of the endoscope 10 to deteriorate, the deterioration of the anti-friction material 17A causes the frictional property of the insertion members to deteriorate.

However, the deterioration of the anti-friction material 17A caused by these radicals can be suppressed in the present embodiments by the radical scavenger 17B.

According to the lubricant layer 17 of the present embodiment and the endoscope 10 including the lubricant layer 17, resistance to gas low-temperature sterilization is improved as described above.

An example of a case where the medical device in which the lubricant for a medical device of the embodiment is used is a medical endoscope has been described in the description of the embodiment. However, the medical device is not limited to an endoscope as long as the medical device is a medical device to be subjected to gas low-temperature sterilization. Examples of the medical device in which the lubricant for a medical device of the embodiment is used include an endoscopic device, an energy device, and the like.

### EXAMPLES

Examples 1 to 8 of the lubricant for a medical device (hereinafter referred to as the lubricant) of the embodiment will be described below together with Comparative Examples 1 and 2.

Table 1 shows the composition and evaluation results of lubricants of Examples 1 to 8 and Comparative Examples 1 and 2. However, the reference numerals of the names of the members will be omitted in Table 1.

TABLE 1

| | Lubricant | | | | | | Evaluation results | | | |
|---|---|---|---|---|---|---|---|---|---|---|
| | | | | | | | Coefficient of dynamic friction | | | |
| | Anti-friction material | | Radical scavenger | | Catalyst | | Before | After | | |
| | | Content | | Content | | Content | sterilization | sterilization | Difference | Comprehensive |
| | Material | (mass %) | Material | (mass %) | Material | (mass %) | test (a) | test (b) | (b − a) | evaluation |
| Example 1 | MoS₂ | 25 | Hydroquinone | 75 | — | — | 0.480 | 0.483 | 0.003 | B |
| Example 2 | MoS₂ | 70 | Hydroquinone | 30 | — | — | 0.450 | 0.465 | 0.015 | A |
| Example 3 | MoS₂ | 90 | Hydroquinone | 10 | — | — | 0.440 | 0.460 | 0.020 | A |
| Example 4 | MoS₂ | 91 | Hydroquinone | 9 | — | — | 0.438 | 0.475 | 0.037 | B |
| Example 5 | MoS₂ | 25 | Benzoquinone | 75 | — | — | 0.479 | 0.485 | 0.006 | B |
| Example 6 | MoS₂ | 40 | Benzoquinone | 60 | — | — | 0.459 | 0.469 | 0.010 | A |
| Example 7 | MoS₂ | 99 | Benzoquinone | 1 | — | — | 0.431 | 0.465 | 0.034 | A |
| Example 8 | MoS₂ | 99.95 | Benzoquinone | 0.05 | — | — | 0.430 | 0.490 | 0.060 | B |
| Comparative | MoS₂ | 100 | — | — | — | — | 0.430 | 0.582 | 0.152 | C |
| Example 1 | | | | | | | | | | |
| Comparative | MoS₂ | 90 | — | — | Pt | 10 | 0.455 | 0.509 | 0.054 | C |
| Example 2 | | | | | | | | | | |

### Example 1

Example 1 is an example of the lubricant layer 17 of the embodiment. As shown in Table 1, molybdenum disulfide (MoS₂) was used as an anti-friction material 17A of a lubricant used in a lubricant layer 17 of Example 1.

The molybdenum disulfide was prepared as powder having a particle size of 10.0 μm or less.

Hydroquinone was used as a radical scavenger 17B of the lubricant.

The hydroquinone was prepared as powder having a particle size of 10.0 μm or less.

The molybdenum disulfide and the hydroquinone were mixed with each other in order to prepare a material to be applied. A mass ratio of the molybdenum disulfide to the hydroquinone was set to 25:75. Accordingly, the material to be applied was prepared.

A planar silicone base material having a size of 100 mm×100 mm was used as an adherend used to form an evaluation sample. A silicone rubber sheet (manufactured by AS ONE Corporation) was used as the silicone base material.

The material to be applied was applied to the silicone base material by a dry method. The thickness of an applied layer was set to 10 μm. Accordingly, an evaluation sample of Example 1 was formed. In the lubricant of this evaluation sample, the content of the hydroquinone in the lubricant layer 17 was set to 75 mass %.

### Examples 2 to 4

An evaluation sample of Example 2 was formed in the same manner as that of Example 1 except that the content of the hydroquinone was set to 30 mass %.

An evaluation sample of Example 3 was formed in the same manner as that of Example 1 except that the content of the hydroquinone was set to 10 mass %.

An evaluation sample of Example 4 was formed in the same manner as that of Example 1 except that the content of the hydroquinone was set to 9 mass %.

### Example 51

In an evaluation sample of Example 5, benzoquinone was used instead of the hydroquinone of Example 1. The content of the benzoquinone in the lubricant layer 17 was set to 75 mass %.

The evaluation sample of Example 5 was manufactured in the same manner as that of Example 1 except that a material to be applied in which molybdenum disulfide and the benzoquinone having the above-mentioned content were mixed with each other was used. The benzoquinone was prepared as powder having a particle size of 10.0 μm or less.

### Examples 6 to 81

An evaluation sample of Example 6 was formed in the same manner as that of Example 5 except that the content of the benzoquinone was set to 60 mass %.

An evaluation sample of Example 7 was formed in the same manner as that of Example 5 except that the content of the benzoquinone was set to 1 mass %.

An evaluation sample of Example 8 was formed in the same manner as that of Example 5 except that the content of the benzoquinone was set to 0.05 mass %.

### Comparative Examples 1 and 2

An evaluation sample of Comparative Example 1 is different from that of Example 1 in that only molybdenum disulfide is used as a lubricant.

Molybdenum disulfide was applied to the same silicone base material as that of Example 1 by a dry method, so that the evaluation sample of Comparative Example 1 was manufactured. The thickness of an applied layer was set to 10 μm.

In an evaluation sample of Comparative Example 2, platinum (Pt) was used instead of the radical scavenger of Example 1. The content of the platinum in the lubricant was set to 10 mass %.

The lubricant of Comparative Example 2 was applied to a silicone base material in the same manner as that of Example 1 except that the composition of a material to be applied was different from that of Example 1.

### [Evaluation]

The evaluation sample of each Example and the evaluation sample of each Comparative Example were subjected to gas low-temperature sterilization 200 times (sterilization test). The gas low-temperature sterilization was performed by a hydrogen peroxide low-temperature plasma sterilization method using STERRAD (registered trademark) NX (registered trademark) (product name; manufactured by Johnson & Johnson K.K.).

The coefficient of dynamic friction of the surface of the evaluation sample to which the lubricant was applied was measured before the sterilization test and after the sterilization test. A surface property tester TRIBIGEAR (registered trademark) TYPE: 14FW (product name; manufactured by Shinto Scientific Co., Ltd.) was used for the measurement of the coefficient of dynamic friction. A stainless steel plate having a thickness of 1 min and a width of 25 mm was used as a counterpart member. Test conditions included a speed of 1000 mm/min, a stroke of 15 mm, 500 times of reciprocation, and an applied load of 500 gf (4.9 N).

A comprehensive evaluation was made as three levels of “very good” (“A” in Table 1). “good” (“B” in Table 1), and “no good” (“C” in Table 1).

A comprehensive evaluation in a case where a coefficient of dynamic friction after sterilization treatment was 0.470 or less was defined as “very good”.

A comprehensive evaluation in a case where a coefficient of dynamic friction after sterilization treatment was higher than 0.470 and lower than 0.500 was defined as “good”.

A comprehensive evaluation in a case where a coefficient of dynamic friction after sterilization treatment was higher than 0.500 was defined as “no good”.

### [Evaluation Result]

As shown in Table 1, the coefficients (a) of dynamic friction of Examples 1 to 8 before the sterilization test (hereinafter simply referred to as the coefficients (a) of dynamic friction) were 0.480, 0.450, 0.440, 0.438, 0.479.0.459, 0.431, and 0.430, respectively. The coefficients (b) of dynamic friction of Examples 1 to 8 after 200 times of the sterilization test (hereinafter simply referred to as the coefficients (b) of dynamic friction) were 0.483, 0.465, 0.460, 0.475.0.485, 0.469, 0.465, and 0.490, respectively.

The coefficients (a) of dynamic friction of Comparative Examples 1 and 2 were 0.430 and 0.455, respectively. The coefficients (b) of dynamic friction of Comparative Examples 1 and 2 were 0.582 and 0.509, respectively.

The coefficients of dynamic friction of all of the respective Examples and the respective Comparative Examples were increased after the sterilization test. It is considered that the reason for this is that the friction characteristics of the lubricants deteriorate due to sterilization treatment.

It is considered that the degree of deterioration of friction characteristics corresponds to the amount of reacting molybdenum disulfide. Accordingly, it is considered that sulfurous acid, sulfuric acid, and the like were generated according to the degree of deterioration of frictional property.

Since the coefficients (b) of dynamic friction of Examples 2, 3, 6, and 7 were 0.470 or less, Examples 2, 3, 6, and 7 were evaluated as “very good”.

Since the coefficients (b) of dynamic friction of Examples 1, 4, 5, and 8 were higher than 0.470 and lower than 0.500. Examples 1, 4, 5, and 8 were evaluated as “god”.

In contrast, both the comprehensive evaluations of Comparative Examples 1 and 2 were “no good”.

In a case where Examples 1 to 4 were compared with each other, the coefficient (a) of dynamic friction was higher as the content of hydroquinone was higher. It is considered that the reason for this is that Examples 1 to 4 had configuration where the content of molybdenum disulfide contributing to frictional property is lower as the content of hydroquinone is higher.

In contrast, the amount of increase in the coefficient (b) of dynamic friction based on the coefficient (a) of dynamic friction (a difference (b-a) in Table 1) was smaller as the content of hydroquinone was higher. It is considered that the reason for this is that the deterioration of molybdenum disulfide was more suppressed as the content of hydroquinone was higher.

Among Examples 1 to 4, the friction characteristics of Examples 2 and 3 were particularly excellent in that the coefficient of dynamic friction was maintained at 0.470 or less even after sterilization treatment was performed 200 times.

In a case where Examples 5 to 8 were compared with each other, the coefficient (a) of dynamic friction was higher as the content of benzoquinone was higher. It is considered that the reason for this is that Examples 5 to 8 had configuration where the content of molybdenum disulfide contributing to friction characteristics is lower as the content of benzoquinone is higher.

In contrast, the amount of increase in the coefficient (b) of dynamic friction based on the coefficient (a) of dynamic friction was smaller as the content of benzoquinone was higher. It is considered that the reason for this is that the deterioration of molybdenum disulfide was more suppressed as the content of benzoquinone was higher.

Among Examples 5 to 8, the frictional property of Examples 6 and 7 were particularly desireable in that the coefficient of dynamic friction was maintained at 0.470 or less even after sterilization treatment was performed 200 times.

In contrast, in Comparative Example 1, the coefficient (a) of dynamic friction was good but the coefficient (b) of dynamic friction thereof significantly deteriorated. As a result, the comprehensive evaluation of Comparative Example 1 was “no good”. It is considered that the amount of change in the coefficient of dynamic friction before and after the sterilization test was significantly increased since the radical scavenger was not contained in the Comparative Example 1.

Even in Comparative Example 2, the coefficient (a) of dynamic friction was good but the coefficient (b) of dynamic friction thereof significantly deteriorated. As a result, the comprehensive evaluation of Comparative Example 2 was “no good”.

In Comparative Example 2, the deterioration of molybdenum disulfide could be suppressed to some extent by a platinum catalyst. However, for example, the amount of change in the coefficient of dynamic friction of Comparative Example 2 was increased 2.7 times in comparison with Example 3 including the same amount of hydroquinone as Comparative Example 2. As a result, the coefficient (b) of dynamic friction of Comparative Example 2 was higher than 0.500.

It was found that hydroquinone was significantly superior to a platinum catalyst in terms of an effect of suppressing the reaction of sterilization gas to molybdenum disulfide.

In a case where the magnitudes of the coefficients (a) of dynamic friction of Comparative Example 2 and Example 3 were compared with each other, the coefficient (a) of dynamic friction of Example 3 was lower than that of Comparative Example 2 by 0.015. A platinum catalyst caused friction characteristics to deteriorate in comparison with hydroquinone as described above in a case where the contents were the same. Accordingly, it is considered that the coefficient (b) of dynamic friction is also increased since the coefficient (a) of dynamic friction is further increased in a case where the content of a platinum catalyst is further increased.

While preferred embodiment of the invention and the respective examples of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A lubricant for a medical device comprising:
an anti-friction material; and
a radical scavenger.

2. The lubricant for a medical device according to claim 1,
wherein the radical scavenger includes at least one of hydroquinone and benzoquinone.

3. The lubricant for a medical device according to claim 1,
wherein the radical scavenger consists essentially of benzoquinone, and
the content of the benzoquinone in the lubricant for a medical device is 0.1 mass % or more and 70 mass % or less.

4. The lubricant for a medical device according to claim 1,
wherein the radical scavenger consists essentially of hydroquinone, and
the content of the hydroquinone in the lubricant for a medical device is 10 mass % or more and 70 mass % or less.

5. A medical device comprising:
the lubricant for a medical device according to claim 1.

6. The medical device according to claim 5,
wherein the medical device is an endoscope that includes a flexible tube having a lumen, and
the lubricant for a medical device is provided in the lumen.
